(19) United States
(12) Patent Application Publication
(10) Pub. No.: US 2009/0317827 A1
(43) Pub. Date: Dec. 24, 2009

(54) Receptor In Dendritic Cells

(75) Inventors: José M. Carballido Herrera, Vienna (AT); Günther Lametschwandtner, Vienna (AT); Gudrun Werner, Vienna (AT); Antal Rot, Vienna (AT)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS AG, Basel (CH)

(21) Appl. No.: 11/913,438

(22) PCT Filed: May 2, 2006
(86) PCT No.: PCT/EP2006/004095

§ 371 Date: Apr. 20, 2009

(30) Foreign Application Priority Data
May 3, 2005 (GB) 0508990.9

(51) Int. Cl. G01N 33/53; C07K 14/705

(52) U.S. Cl. 435/7.2; 530/350

(57) ABSTRACT

The present invention provides the use of GPR91 as a target in dendritic cells.

The present invention relates to GPR91 as a target in dendritic cells.

G protein-coupled receptors (GPRs) constitute a major class of proteins responsible for transducing a signal within a cell and represent one of the largest families of proteins with over 350 reported genes in the human genome.

GPRs have 3 structural domains: an amino terminal extracellular domain, a transmembrane domain containing 7 transmembrane segments, 3 extracellular loops and 3 intracellular loops and a carboxy terminal intracellular domain. Upon binding of a ligand to an extracellular portion of a GPR, a signal is transduced within the cell that results in a change in a biological or physiological property of the cell. GPRs, along with G-proteins and effectors such as e.g. intracellular enzymes and channels modulated by G-proteins, are the components of a modular signaling system that connects the state of intracellular second messengers to extracellular inputs.

GPRs therefore are a major target for drug action and development. As a novel GPR, the G protein-coupled receptor GPR91 was identified (see e.g. Wittenberger T. et al., J. Mol. Biol. 2001, 307, 799-813). Little is known about the function of GPR91.

Surprisingly we have now found that GPR91 is mainly expressed in dendritic cells and only to a small extent. if even, in monocytes, T-cells or B-cells.

In several aspects the present invention provides

1. GPR91 for use, e.g., or the use of GPR91

### 1.1 As a Target in Dendritic Cells;

1.2 For Diagnosing in Dendritic Cells Disorders Mediated, e.g. Associated with, e.g. Driven, by GPR91 Activity.

Disorders, e.g. including diseases, mediated, e.g. associated with, e.g. driven, by GPR91 activity are believed to include immune, such as autoimmune disorders, inflammatory disorders, allergic disorders, infectious diseases, cardiovascular disorders, cancer, disorders associated with transplantation, neurodegenerative disorders; more specifically disorders including mast cell mediated diseases such as allergic and nonallergic asthma, chronic obstructive pulmonary disease (COPD), allergic rhinitis, anaphylaxis, allergic gastrointestinal disease, atopic dermatitis, rheumatoid arthritis, psoriasis and other allergic, autoimmune and inflammatory diseases; immunological disorders including arthritis, asthma, immunodeficiency diseases such as AIDS, renovascular hypertension, a disease closely linked to atherosclerosis, diabetes and renal failure, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, autoimmunity disorders, such as autoimmune infertility, lense tissue injury, demyelination, systemic lupus erythematosis, drug induced hemolytic anemia, Sjogren's disease, and scleroderma, cardiovascular disorders, dermatological disorders, metabolic diseases, cancer disorders, e.g. leukemia, gastrointestinal and liver diseases, hematological disorders, reproduction disorders, diseases of the endocrine system, inflammatory diseases, muscle-skeleton disorders, neurological disorders, urological disorders, respiratory diseases, disorders associated with infections such as bacterial, fungal, protozoan, and viral infections, particularly those caused by HIV viruses, cardiovascular diseases including acute heart failure, hypotension, hypertension, angina pectoris, myocardial infarction, hematological diseases, genito-urinary diseases including urinary incontinence and benign prostate hyperplasia, osteoporosis, neurodegenerative disorders, such as peripheral and central nervous system disorders including pain, Alzheimer's disease and Parkinson's disease, metabolic diseases, gastro-enterological diseases, diseases of muscles or the skeleton, immunological diseases, developmental diseases or diseases of the reproductive system, e.g. diseases associated with kidney, brain, heart or lung disorders.

Disorders and diseases for which GRPR91 may be used according to the present invention include specifically psoriasis, rheumatoid arthritis, disorders or diseases associated with transplantation, disorders or diseases associated with cancer, lupus, e.g. systemic lupus erythematosis, inflammatory bowel disease, atopic dermatitis, asthma, cardiovascular disorders, such as psoriasis and conditions which arise in a patient having psoriasis, e.g. psoriatic arthritis.

Psoriasis is an autoimmune disorder which is related with Th1 cell activation and which is associated with inflammatory skin disease in which skin cells replicate at an extremely rapid rate. New skin cells are produced about 8 times faster than normal—over several days instead of a month—but the rate at which old cells slough off remains unchanged. This causes cells to build up on the skin's surface, forming thick patches, or plaques, of red scores (lesions) covered by flaky, silvery-white dead skin cells (scales).

Rarely life-threatening, at its mildest, psoriasis can be itchy and sore. At its worst, it's painful, disfiguring and debilitating. About ⅔s of the people with psoriasis have a mild form of the disease. About ⅓ have moderate or severe psoriasis. Psoriasis can affect people at any age, but it most often strikes those between the ages of 15 and 35.

There are 5 forms of psoriasis. Plaque psoriasis is the most common—affecting 4 out of 5 people who have psoriasis. Plaque psoriasis may start with small reed bumps and progress to larger lesions.

The plaques of psoriasis occur most frequently on the elbows, knees, other parts of the legs, scalp, back, face, palms and sole of the feet. Psoriasis can also affect the fingernails and toenails, causing pitting, discoloration or tissue buildup around the nails.

According to the National Institute of Arthritis and Musculoskeletal and Skin Diseases, about 15% of people with psoriasis also get psoriatic arthritis, which can be progressively disabling if untreated.

It is believed that T lymphocytes (T cells) play an important role in psoriasis and it was found that Th1 cell activation plays a major role. Psoriasis also has a genetic component: in about ⅓ of psoriasis cases, there is a family history of the disease.

T cells circulate throughout the body, orchestrating the immune system's response to foreign invaders like bacteria or viruses. In people with psoriasis, the defective T cells are overactive and migrate to the skin as if to heal a wound or ward of an infection. This process leads to the rapid growth of skin cells, triggering inflammation and development of lesions. Until now, no single test exists to diagnose psoriasis, but a dermatologist can usually determine it by appearance of the skin and by locking at an individual's personal and family medical history.

A dermatologist can usually determine psoriasis by appearance of the skin and by locking at an individual's personal and family medical history, but, until now, no single test exists to diagnose psoriasis.

The use of GPR91 as a biomarker for a use as claimed in any one of claims 1 or 2.

In several other aspects the present invention provides
2. GPR91, e.g. or the Use of GPR91, as a Biomarker, for a Use as Indicated Under 1.1 or 1.2 Above,
   e.g. in a sample of an individual,
   e.g. in a sample of a body fluid or a tissue sample of an individual,
   e.g. in a biopsy-sample of an individual,
   e.g. skin biopsy-sample, of an individual.

GPR91 as indicated under any of 1. or 2 above includes GPR91 in dendritic cells in any form, e.g. in the form of
a nucleic acid encoding GPR91, e.g. including a nucleic acid encoding a derivative of GPR91,
GPR91 protein, e.g. including protein which is a GPR91 derivative, or
GPR91 secreting cells, e.g. or a derivative of GPR91 secreting cells.

“A derivative” of GPR91 nucleic acid or protein, e.g. in secreting cells, according to the present invention includes a fragment, a mutant, a variant, an homolog or a modification of a GPR91 protein, or of a nucleic acid encoding GPR91, which retains, e.g. essentially, the biological function of GPR91, e.g. which retains, e.g. essentially, the biological function of GPR91 in dendritic cells.

GPR91-secreting cells, e.g. including GPR91 producing cells, include antigen presenting cells (APC), such as dendritic cells (DC).

Thus, GPR91 for use as provided by the present invention includes splice variants encoded by mRNA generated by alternative splicing of a primary transcript, amino acid mutants, posttranslational modifications, such as glycosylation and phosphorylation variants, and modifications which are covalent derivatives of GPR91 and which retain the biological function of GPR91 in dendritic cells. Exemplary GPR91 derivatives include modifications wherein the GPR91 protein is covalently modified by substitution, e.g. substitution originating from appropriate means, e.g. chemical or enzymatic means, by a moiety in the GPR91 protein. Such a moiety e.g. includes one or more amino acids, e.g. naturally occurring amino acids and other than naturally occurring amino acids, and/or a detectable moiety. A detectable moiety includes an enzyme, a radioisotope, tags, toxins and genes such as oncogenes and tumour suppressor genes. GPR91 derivatives further include naturally occurring variants of GPR91, e.g. provided within a particular species. Such a variant may be encoded by a related gene of the same gene family, by an allelic variant of a particular gene, or represent an alternative splicing variant of the GPR91 gene.

A GPR91 derivative as used herein also includes fragments of a nucleic acid encoding GPR91, or of the GPR91 protein, and comprises individual GPR91 domains and smaller polypeptides derived from GPR91 domains. Preferably, smaller polypeptides derived from GPR91 according to the invention define a single functional activity which is characteristic of GPR91. Fragments may in theory be of almost any size, as long as they retain the biological characteristic of GPR91. Preferably, fragments will be between 12 and 210 nucleic acids in length or between 4 and 70 amino acids, respectively. Longer fragments are regarded as truncations of the full-length GPR91.

Derivatives of GPR91 as used herein also comprise mutants thereof, which may contain amino acid deletions, additions or substitutions, subject to the requirement to retain the biological function of GPR91 in dendritic cells. Conservative amino acid substitutions may be made substantially without altering the nature of GPR91, e.g. by truncations from the 5′ or 3′ ends. Deletions and substitutions also include deletions and substitutions in fragments of GPR91. GPR91 mutants may be produced from a DNA encoding GPR91 which has been subjected to in vitro mutagenesis resulting e.g. in an addition, exchange and/or deletion of one or more amino acids in GPR91. For example, substitutional, deletional or insertional variants of GPR91 can be prepared by recombinant methods and screened for functional similarity to the native forms of GPR91.

Derivatives of GPR91 as used herein also include GPR91 homologs, preferably GPR91 homologs retain substantial homology with GPR91. As used herein, “homology” means that GPR91 and a GPR91 homolog share sufficient characteristics to retain the biological function of GPR91 in dendritic cells. Preferably, homology is used to refer to sequence identity. Thus, the derivatives of GPR91 preferably retain substantial sequence identity with the nucleic acid sequence as given in

“Substantial homology”, where homology indicates sequence identity, means more than 50% sequence identity, preferably more than 75% sequence identity and even more preferably a sequence identity of 80% and more, e.g. 90% and more, such as 90%, 91%, 92%, 93%, 94%, 95%, 96%, 97%, 98% or 99%.

Preferably GPR91 is originating from a mammal in dendritic cells.

The nucleic acid encoding GPR91 preferably has the nucleic acid sequence as described in Wittenberger T. et al., J. Mol. Biol. 2001, 307, 799-813). The GPR91 protein preferably corresponds to the translated protein sequence of the above mentioned nucleic acid.

Biomarker as used herein means that determination (=detection and/or quantification) of a GPR91 molecule in dendritic cells of a sample of an individual is an indicator for a disorder or disease as such and/or is useful for monitoring the status of a disorder or disease related with GPR91 activity.

In another aspect the present invention provides a method for diagnosing a disorder or disease which is mediated, e.g. associated with, e.g. driven by, GPR91 activity, comprising
a) providing a sample of an individual,
b) determining the level of GPR91 in dendritic cells in said sample,
c) comparing the level of GPR91 as determined in step b) with a reference level from a sample of a healthy control individual in dendritic cells, and
d) diagnosing a disorder or disease which is mediated, e.g. associated with, e.g. driven by, GPR91 activity by determination whether the level of GPR91 as determined in step b) is, e.g. significantly, different from said reference level.

In another aspect the present invention provides a method for monitoring the therapeutic efficacy in the treatment of an individual with a substance which is expected to have an effect on reducing or curing a disorder or disease which is mediated, e.g. associated with, e.g. driven by GPR91 activity, which method comprises determining the level of GPR91 in dendritic cells in a sample of said individual and comparing that level with the level of GPR91 prior to administration of said substance.

A sample of an individual according to a use or a method of the present invention includes a sample of a body fluid or a tissue sample. A body fluid may be derived e.g. from blood, e.g. including isolated mononuclear cells, or from a blood fraction, e.g. including plasma or serum, preferably serum. A tissue sample may be a biopsy, e.g. such as a skin biopsy.

In another aspect the present invention provides the a use or a method of the present invention wherein a sample is a body fluid or a tissue sample of an individual, e.g. a body fluid may be derived from blood, e.g. isolated dendritic cells, or from a blood fraction, e.g. plasma or serum, e.g. serum; e.g. the tissue sample may be a biopsy, e.g. such as a skin biopsy.

Dendritic cells from a sample of an individual may be isolated as appropriate, e.g. according, e.g. analogously, to a method as conventional.

Detection means in dendritic cells for determining the level of GPR91 include means as conventional, e.g. immunoassays, such as an immunodiagnostic method, an enzyme linked immunoassay (ELISAs); a fluorescence based assay, such as dissociation enhanced lanthanide fluoroimmunoassay (DELFIA), an radiometric assay or by carrying out a GPR91 specific Polymerase Chain Reaction (PCR); specifically detection means include a molecule which specifically recognizes GPR91, e.g. a molecule which is directly or indirectly detectable, preferably comprising an antibody, including antibody derivatives or fragments thereof, e.g. an antibody which recognizes GPR91, e.g. a label bearing GPR91 recognizing antibody.

Such label may be a conventional label, e.g. biotin or an enzyme such as alkaline phosphatase (AP), horse radish peroxidase (HRP) or peroxidase (POD) or a fluorescent molecule, e.g. a fluorescent dye, such as e.g. fluorescein isothiocyanate. Preferably the label is biotin. The label bearing molecule, e.g. the label bearing antibody, may be detected according to methods as conventional, e.g. via fluorescence measurement or enzyme detection methods.

An antibody fragment or antibody derivative includes a fragment or a derivative, e.g. chemically or enzymatically modified, of an antibody which still is capable of recognising GPR91.

GPR91-secreting cells in a sample of a body fluid of an individual, e.g. blood, may be determined by a method as conventional, e.g. by the following method:

Dendritic cells may be purified, e.g. separated by a density gradient, from the sample, e.g. blood, and the purified cells obtained are stained. Anti-GPR91 antibodies, e.g. fluorescence labeled anti-GPR91 antibodies, are added to the stained cell preparation, optionally after stimulation of the cells, e.g. with interleukin-4, and the level of GPR91-secreting cells is determined.

Optionally, GPR91 comprised in the sample or the GPR91 recognizing, e.g. detectable, molecule comprised in the detection means is immobilized on a solid phase. An appropriate solid phase includes e.g. conventional solid phases used for immobilization, e.g. a plastic plate like a polystyrene or polyvinyl plate, especially a microtiter plate. Also microbeads can be used as a solid phase, e.g. coated microbeads. The solid phase can be coated with a coating material the nature of which depends e.g. on the label comprised in the detection means. The coating material should be able to bind to the label, e.g. if the label is biotin a coating material includes streptavidin, e.g. covalently bound to the solid phase.

Preferably determination of GPR91 in dendritic cells is carried out by using a molecule which specifically recognizes the GPR91, e.g. an antibody, an antibody derivative, or an antibody fragment, such as an anti GPR91 antibody, e.g. a commercially available GPR91 specific antibody. Detection of GPR91-antibody formation preferably is carried by an immunodiagnostic assay method.

In another aspect the present invention provides a method for diagnosing a disorder or disease which is mediated, e.g. associated with, e.g. driven, by GPR91 activity according to the present invention wherein the level of GPR91 in dendritic cells is determined by use of an GPR91 specific antibody.

In another aspect the present invention provides a kit for diagnosing of a disorder or disease which is mediated, e.g. associated with, e.g. driven, by GPR91 activity in a sample of an individual comprising
a) a molecule which recognizes GPR91, optionally in a labeled form,

b) instructions how to use said kit in dendritic cells,
c) optionally detection means,
d) optionally a solid phase.

Such kit may further comprise a substantial component, e.g. including an appropriate environment of a sample to be tested and, e.g. appropriate means to determine GPR91 in a sample to be tested.

In a further aspect the present invention provides an assay for identifying an agent that modulates GPR91 activity, comprising
a) determining the level of GPR91 in dendritic cells of a sample of an individual, in the absence and in the presence of a candidate compound which is expected to modulate the level of GPR91,
b) identifying a candidate compound which modulates the level of GPR91 as determined in step a) as an agent, e.g. and
c) using such agent as a pharmaceutical in the treatment of disorders or diseases mediated, e.g. associated with, e.g. driven, by GPR91 activity.

The level of GPR91 is determined as appropriate, e.g. as described herein.

A candidate compound as described herein is a compound which may be expected to modulate the level of GPR91, or GPR91 activity or GPR91 secreting cells, and includes compound(s)(libraries) from which its influence on GPR91 can be determined. Compound (libraries) include for example oligopeptides, polypeptides, proteins, antibodies, mimetics, small molecules, e.g. low molecular weight compounds (LMW's).

An agent is a candidate compound which modulates the level of the level of GPR91, or GPR91 activity or GPR91 secreting cells, e.g. in dendritic cells in a sample form a patient, e.g. a blood sample, such as serum, or a skin biopsy. An agent includes oligopeptides, polypeptides, proteins, antibodies, mimetics, small molecules, e.g. low molecular weight compounds (LMW's).

In another aspect the present invention provides an agent identified by an assay or a method of the present invention.

An agent of the present invention may exhibit pharmacological activity and is therefore useful as a pharmaceutical. An agent of the present invention may show therapeutic activity, e.g. in disorders or diseases mediated, e.g. associated with, e.g. driven by GPR91 activity.

In another aspect the present invention provides the use of an agent of the present invention as a pharmaceutical in disorders mediated, e.g. associated with, e.g. driven by GPR91 activity.

For pharmaceutical use an agent of the present invention for treatment includes one or more, preferably one, agent of the present invention, e.g. a combination of two or more agents of the present invention.

In another aspect the present invention provides the use of an agent of the present invention for the manufacture of a medicament for the treatment of disorders or diseases mediated, e.g. associated with, e.g. driven by GPR91 activity.

In another aspect the present invention provides a pharmaceutical composition comprising an agent of the present invention beside at least one pharmaceutical excipient, e.g. appropriate carrier and/or diluent, e.g. including fillers, binders, disintegrators, flow conditioners, lubricants, sugars and sweeteners, fragrances, preservatives, stabilizers, wetting agents and/or emulsifiers, solubilizers, salts for regulating osmotic pressure and/or buffers.

In another aspect the present invention provides a method for the treatment of disorders or diseases mediated, e.g. associated with, e.g. driven, by GPR91 activity, comprising administering an effective amount of an agent of the present invention to a subject in need of such treatment.

For such treatment, the appropriate dosage will, of course, vary depending upon, for example, the chemical nature and the pharmacokinetic data of a compound of the present invention used, the individual host, the mode of administration and the nature and severity of the conditions being treated. However, in general, for satisfactory results in larger mammals, for example humans, an indicated daily dosage includes a range
from about 0.001 g to about 1.5 g, such as 0.001 g to 1.5 g;
from about 0.01 mg/kg body weight to about 20 mg/kg body weight, such as 0.01 mg/kg body weight to 20 mg/kg body weight,
   for example administered in divided doses up to four times a day.

An agent of the present invention may be administered by any conventional route, for example enterally, e.g. including nasal, buccal, rectal, oral, administration; parenterally, e.g. including intravenous, intramuscular, subcutaneous administration; or topically; e.g. including epicutaneous, intranasal, intratracheal administration; via medical devices for local delivery, e.g. stents,

e.g. in form of coated or uncoated tablets, capsules, (injectable) solutions, solid solutions, suspensions, dispersions, solid dispersions; e.g. in the form of ampoules, vials, in the form of creams, gels, pastes, inhaler powder, foams, tinctures, lip sticks, drops, sprays, or in the form of suppositories.

For topical use, e.g. including administration to the eye, satisfactory results may be obtained with local administration of a 0.5-10%, such as 1-3% concentration of active substance several times daily, e.g. 2 to 5 times daily.

An agent of the present invention may be administered in the form of a pharmaceutically acceptable salt, e.g. an acid addition salt or metal salt; or in free form; optionally in the form of a solvate. An agent of the present invention in the form of a salt may exhibit the same order of activity as an agent of the present invention in free form; optionally in the form of a solvate.

An agent of the present invention may be used for pharmaceutical treatment according to the present invention alone, or in combination with one or more other pharmaceutically active agents.

Combinations include fixed combinations, in which two or more pharmaceutically active agents are in the same formulation; kits, in which two or more pharmaceutically active agents in separate formulations are sold in the same package, e.g. with instruction for co-administration; and free combinations in which the pharmaceutically active agents are packaged separately, but instruction for simultaneous or sequential administration are given.

### DESCRIPTION OF THE FIGURES

FIG. 1 shows the relative expression of GPR91 in various immune cells. From FIG. 1 it is immediately evident that GPR91 is mainly expressed in untreated dendritic cells and only very limited, if even, in monocytes, T-cells or B-cells.

FIG. 2 shows the relative expression of GPR91 in various tissues.

The following abbreviations are used:

DC dendritic cells
hPBCs human peripheral blood cells
hPBTC human peripheral blood T cells
hPBM human peripheral blood monocytes
IL-10 interleukin-10
LPS lipopolysaccharide
PCR polymerase chain reaction
PHA phytohaemagglutinin
RT-PCR reverse transcriptase polymerase chain reaction

### EXAMPLE

### Gene Expression of GPR91 by Quantitative Real-Time PCR

### a) RT-PCR Protocol

Primers are designed to GPR91 sequences and to the housekeeping gene EF1α as internal control using the PrimerExpress program (Applied Biosystems).

The amplification procedure follows state of the art protocols for quantitative real-time PCR (TaqMan). Each reaction mixture contains 1 Taqman Universal Master Mix (Applied Biosystems), 0.25 Units Platinum Taq DNA polymerase (Gibco) 50 ng cDNA, and the primers at 450 nM (forward and reverse primer) and 200 nM (TaqMan probe) concentrations in a final volume of 25 μL. Cycling is carried out in a 96-well optical reaction plate (Applied Biosystems) using an ABI7700 PCR instrument. Cycling conditions are as follows: Pre-activation at 50° for 2 minutes, denaturation at 94° for 10 minutes for 1 cycle, then 45 cycles of denaturation at 95° for 15 seconds, annealing and extension for 1 minute at 60°. The kinetics of the reaction is recorded at 488 nm excitation and 518 nm emission wavelengths.

b) Preparation of Cells, RNA and First cDNA Synthesis

Cells used in the analysis are primary, human monocytes (hPBMs) activated by LPS, by IL-10 or a combination of both, primary human T cells (hPBTCs) activated by PHA, primary human B-cells (hPBCs) activated by anti-CD40 ligation, human monocyte derived dendritic (DCs) activated by LPS, IL-10 or a combination of both.

Primary human monocytes are isolated by elutriation according to standard protocols. T- and B-cells are isolated from peripheral blood by MACS beads (Milteny) according to manufacturer's protocols. Human DCs are generated in-vitro by culturing hPBMs in the presence of GM-CSF and IL-4 according to standard protocols.

RNA is prepared from cells using SNAP™ extraction kits (InVitrogen) according to the manufacturer's protocol.

First strand cDNA is prepared from total cellular RNA using the TaqMan Reverse Transcription kit including random hexamer primers from Applied Biosystems.

### c) Gene Expression Profiles

GPR91 sequences represented by primers provided as indicated above are amplified in commercially available cDNA derived from tissues (Clontech panel I and II) and in the different cell types described above by quantitative RT-PCR using reagents supplied by Applied Biosystems and following the manufacturer's protocol. Control reactions are performed with primers specific for the housekeeping gene EF1α.

In more detail:

Selected sequences are profiled in cDNA derived from tissues and in the different cell types as described above by quantitative RT-PCR following manufacturer's protocol or semiquantitative PCR. Control reactions are performed with primers specific for the housekeeping gene EF1α. Tissue cDNAs used for RT-PCR profiling are purchased from Clontech. In semiquantitative PCR, each reaction mixture contains 0.2 mM dNTP's, 1×PCR buffer containing 1.5 mM MgCl₂, 0.5 Units Taq DNA polymerase, 50 pmol each primer and deionized H₂O in a total volume of 25 μl.

Template cDNA used in either from commercial cDNA derived from tissue samples from Clontech panel I and II (2.5 μl) or form cDNA's prepared from cell types as described above (1 μl). Cycling is carried out in 0.2 ml tubes using a Biometra Trio PCR machine with the optimally determined annealing temperature. Cycling conditions are as follows: denaturation at 94° for 1 minute, 45 seconds for 1 cycle, then 35 cycles of denaturation at 94° for 15 seconds, annealing for 15 seconds and extension at 72° for 30 seconds. Reactions are analysed on a 1.5% agarose gel and stained with ethidium bromide. Control RT-PCR reactions are performed with primers specific to the housekeeping gene EF1α. Expression values of GPR91 are normalized to the expression of EF1α according to:

Relative Expression N=1000×2^{(CT}^{_{—}}^{PSI-CT}^{_{—}}^{EF1α)}

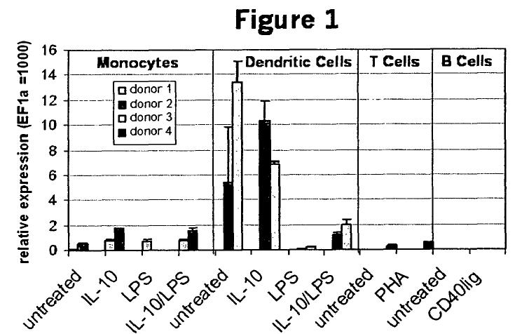

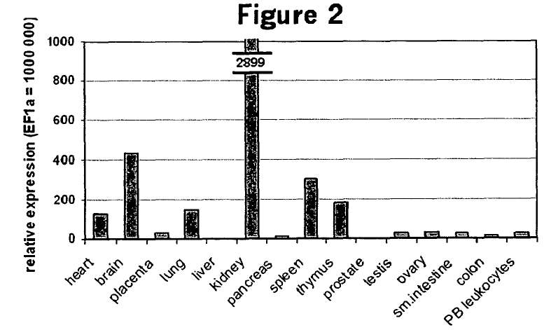

Results are as set out in FIGS. 1 and 2.

1. The use of GPR91 as a target in dendritic cells.

2. The use of GPR91 for diagnosing in dendritic cells disorders mediated by GPR91 activity.

3. The use of GPR91 as a biomarker for a use as claimed in claim 1

4. A method for diagnosing a disorder or disease which is mediated by GPR91 wherein the level of GPR91 in dendritic cells is determined by use of an GPR9I specific antibody.

5. A kit for diagnosing of a disorder or disease which is mediated by GPR91 in a sample of an individual comprising
a) a molecule which recognizes GPR91, optionally in a labeled form,
b) instructions how to use said kit in dendritic cells,
c) optionally detection means,
d) optionally a solid phase.
